(19) United States
(12) Patent Application Publication
(10) Pub. No.: US 2007/0292480 A1
(43) Pub. Date: Dec. 20, 2007

(54) Delivery System for Ingestible Components

(75) Inventors: Shi-Qiu Zhang, Tenafly, NJ (US); Stephanie Luther-Forsstrom, Monroe, NY (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: CONOPCO, INC., D/B/A UNILEVER, Englewood Cliffs, NJ (US)

(21) Appl. No.: 11/424,124

(22) Filed: Jun. 14, 2006

(51) Int. Cl. A61K 47/00; A61K 36/82; A61K 31/7048; A61K 31/522; A61K 31/353

(52) U.S. Cl. 424/439; 424/729; 514/263.31; 514/27; 514/456

(57) ABSTRACT

A delivery system for ingestible components is described. The delivery system comprises at least one orally soluble film but often comprises more than one orally soluble film. The orally soluble films within the delivery system comprise a different biologically active component or mixture of biologically active components so that a consumer can ingest more than one active at a single time. Often, the biologically active components are agents isolated from tea.

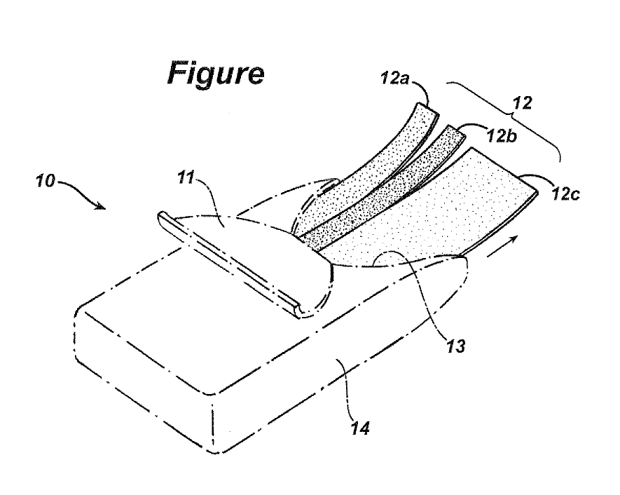

### FIELD OF THE INVENTION

The present invention is directed to a delivery system for ingestable components. More particularly, the invention is directed to a delivery system for ingestable components comprising at least one water soluble film wherein the film is suitable to dissolve in a mouth of a consumer. The delivery system for ingestable components of this invention can be used to deliver a biologically active component like an amino acid, protein, saccharide, antioxidant, alkaloid, medication, mixture thereof or the like. Moreover, the delivery system for ingestable components of this invention can optionally be formulated to taste like a candy, beverage or food product and can enhance the condition of a consumer, medicate the consumer, and/or deliver an antioxidant benefit to a consumer that consumes the same. In a preferred embodiment, the delivery system for ingestable components comprises a component naturally found in tea, like, for example, a polyphenol (i.e., antioxidant) selected from thearubigin, theaflavins, theanine and/or epigallocatechin gallate. In yet another preferred embodiment, the delivery system for ingestible components of this invention comprises more than one water soluble film.

### BACKGROUND OF THE INVENTION

Beverage components, especially those found in tea, like Lipton® Tea, have been associated with many positive health benefits. For example, tea antioxidants are highly desirable because studies show they can reduce cholesterol levels and prevent certain cancers. Other studies show that amino acids, like L-theanine can reduce stress, lower blood pressure, heighten mental acuity, promote concentration and improve learning performance.

Unfortunately, in order to consume such desired components, consumers are often required to ingest a food or beverage product (i.e., like a glass of tea and/or a meal with mushrooms) that is rich in at least one of these types of components. During the course of a hectic day, it is not always possible for the busy consumer to have access to a meal and/or beverage that comprises such desired components. For example, in the middle of traffic, during a lecture, or while taking an exam, it would be beneficial to the consumer to have a convenient delivery system that is suitable to deliver desired ingestable components; especially, those that enhance the condition or performance of a consumer and are beneficial to one's health.

Similarly, consumers often are required to take medication during the day, and ingesting the same (for example, in pill form) is not always convenient during the course of daily activities.

It is of increasing interest to develop a convenient delivery system for ingestible components. This invention, therefore, is directed to a delivery system suitable to deliver a biologically active component like, for example, an amino acid, protein, saccharide, antioxidant, alkaloid, medication, mixture thereof or the like. The delivery system, unexpectedly, can be used to, for example, enhance the condition of a consumer, medicate the consumer and/or deliver an antioxidant benefit without requiring the consumption of a pill, meal, beverage, or the like.

### ADDITIONAL INFORMATION

Efforts that utilize water soluble edible films have been disclosed. In U.S. Application Publication No. 2005/0008735 A1, edible chocolate snacks made with polymer and chocolate are described.

Other efforts have been disclosed for making anti-oxidant solutions. In U.S. Pat. No. 5,527,552, water-soluble and fat-insoluble polyphenolic anti-oxidants present in green tea are made into solution in an edible non-ionic lipid-soluble solvent.

Still other efforts have been disclosed for making products with green tea extract. In U.S. Patent Application No. 2003/0235630 A1, dental hygiene products with an edible film are described.

Even other efforts have been disclosed for making dissolvable films. In U.S. Patent Application No. 2005/0186257 A1, dissolvable films and methods for their manufacture are disclosed.

None of the additional information above describes a delivery system for delivering, in an edible film, ingestible components that can, for example, enhance the condition of a consumer, medicate the consumer, and/or deliver an antioxidant benefit to a consumer that consumes the same when the delivery system comprises at least one edible and water soluble film and a biologically active component that makes up from about 0.015 to about 35% by weight of the total weight of the delivery system.

### SUMMARY OF THE INVENTION

In a first aspect, the present invention is directed to a delivery system for ingestible components, the delivery system comprising:
(a) at least one edible and water soluble film; and
(b) at least one biologically active component
   wherein the biologically active component makes up from about 0.015 to about 35 percent by weight of the total weight of the delivery system.

In a second aspect, the present invention is directed to a method for delivering an ingestible component to a consumer with the delivery system of the first aspect of this invention.

In a third aspect, the present invention is directed to a package for dispensing the delivery system of this invention.

Biologically active, as used herein, means suitable to improve the physiological and/or physical state of a human subsequent to ingesting and, for example, suitable to enhance the mental condition of a consumer, suitable to have a medicinal impact (like lower blood cholesterol levels), suitable to deliver an antioxidant benefit or a combination thereof. Delivery system, as used herein, means a system with a film suitable to dissolve in the mouth of a consumer (i.e., orally soluble) and comprising ingestible components like biologically active components and optional additives such as flavors and colors. As used herein, vitamins are not meant to be included in the definition of a biologically active component but can be included as optional additives. More than one film, as used herein, means at least two films that do not have an identical formulation (i.e., not the same biologically active component or mixture of biologically active components) notwithstanding the fact that the water or orally soluble film may be the same. More than a single film or each film, therefore, does not mean two or more identical films. For reasons of brevity, therefore, a delivery system comprising 200 films with only biologically active component A, and 200 films with only biologically active component B would be a 2 film delivery system. Water soluble film, orally soluble film and film, as used herein, are meant to be the same type of film.

### BRIEF DESCRIPTION OF THE DRAWING

The subject matter which is regarded as the invention is particularly pointed out and distinctly claimed in the concluding portion of the specification. The invention, however, may be best understood by reference to the following description taken in conjunction with the accompanying drawing FIGURE in which:

The FIGURE illustrates and depicts one form in which the delivery system for ingestible components of this invention may take, and particularly, illustrates a form whereby the delivery system comprises three (3) films that are side-by-side.

### DETAILED DESCRIPTION OF THE PREFERRED EMBODIMENTS

The only limitation with respect to the type of edible and water soluble film that may be used in this invention is that the film is suitable for consumption by humans and orally soluble. Illustrative examples of the type of edible films that may be used in this invention include those which comprise pullulan, hydroxypropylmethyl cellulose, hydroxyethylcellulose, hydroxypropyl cellulose, polyvinyl alcohol, polyvinyl pyrrolidone, carboxymethyl cellulose, or copolymers or blends thereof.

Other types of films that may be used in this invention include those comprising sodium alginate, polyethylene glycol, xanthan gum, tragacanth gum, guar gum, acacia gum, arabic gum, polyacrylic acid, methylmethacrylate, carboxyvinyl polymer, amylose, dextrin, pectin, chiten, chitosan, levan, elsinan, collagen, gelatin, zien, whey, soy, gluten mixtures thereof or the like.

Even other agents that may be used to make the films of the present invention include microcrystalline cellulose, corn and potato starches, flours, seaweed extracts (e.g., carrageenans) or the like. In a preferred embodiment, however, the edible and water soluble film used in this invention is one which comprises at least about 15 to about 95%, and preferably, from about 30 to about 85%, and most preferably, from about 50 to about 75% by weight pectin, based on total weight of the delivery system and including all ranges subsumed therein.

As to the biologically active component that may be used in this invention, the same is limited only to the extent that it is an agent suitable to improve the condition, including mental, physiological, and/or physical state of a human subsequent to ingesting.

Illustrative and non-limiting examples of the types of biologically active components that may be used in this invention include tea (Camillia sinesis) polyphenols like thearubigin, theaflavin, epigallocatechin gallate, epigallocatechin, epicatechin, epicatechin gallate and mixtures thereof. Other suitable biologically active components that may be used in this invention include alkaloids like, for example, caffeine, theobromine and mixtures thereof. Even other suitable biological actives that may be used in this invention include amino acids, preferably theanine which is found, for example, in the sencha of the leaves of a tea plant.

Still other biologically active components suitable for use in this invention include anti-histamines (like brompheniramine maleate and chlorpheniramine maleate); decongestants (like phenylepherine and pseudoephedrine hydrochloride); anti-tussive agents (like benzonatate and caramiphen edisylate); antimicrobial agents (like triclosan and cetyl pyridium chloride); expectorants (like ipecac and potassium iodide); anti-diarrheals (like loperamide and an opiate); psychopharmacological drugs (like chlorpromazine and methotrimeprazine); pharmacological drugs (like Lipitor®, Plavix®, Nexium®, Advair™, and Prevacid®); antiparkinsonism drugs (like levodopa and amantadine), antibiotics, over the counter agents (like aspirin and ibuprophen), as well as other actives that are generally known to improve the mental, physiological and/or physical state of humans after being administered.

It is particularly noted that with respect to combinations of biologically active components that can be used on a single film in the delivery system of this invention, any combination is possible as long as the delivery system that is prepared and the actives used therewith are stable enough to be effective and ingested. In a preferred embodiment, the delivery system for ingestible components of this invention comprises from about 0.2 to about 25%, and most preferably, from about 0.3 to about 12% by weight biologically active component, based on total weight of the delivery system and including all ranges subsumed therein.

The delivery system of the present invention preferably comprises at least two water soluble (i.e., orally soluble) films, and most preferably, at least three water soluble films. The films themselves within the delivery system for ingestible components of this invention may be made of the same or different material or agents, but preferably are made of the same material. Thus, for example, if one water soluble film comprises about 68% by weight pectin, it is preferred that the additional films be prepared from the same material.

In the present invention it has been discovered that one water soluble film within the delivery system for ingestible components of this invention may contain a first biologically active component and a second and third film, for example, may contain a second and third biologically active component, respectively.

Of course, it is within the scope of this invention to employ only a single water soluble film and to have the single film contain all of the desired biologically active components. A single film can be desired when, for example, the biologically active components are isolated from tea and supplied in a tea powder, or tea cream (i.e., by-product black tea components resulting from ready-to-drink tea making processes which usually contain thearubigin, theaflavin, caffeine, polysaccharides and protein). The preferred pH of the film is typically from about 3.5 to about 6.5 when one film is desired and the biologically active component is supplied from a tea powder and/or cream. Often, however, different biologically active components have different dosage, stability and reactivity requirements and/or characteristics. In view of this, and in a preferred embodiment, it is often desirable for each film within the delivery system of the present invention to contain a different biologically active component. In an especially preferred embodiment, the delivery system of the present invention comprises a film with polyphenol, and a second film with alkaloid and amino acid, or two additional separate films, the second with alkaloid and the third with amino acid, whereby a delivery system comprising all three films is often the most desired.

When preparing the delivery system for ingestible components comprising more than one film, each film within the delivery system (again, preferably made of the same orally soluble material) is treated to have a pH that is suited for the particular biologically active component it is carrying. Therefore, in the application where the biologically active components are isolated from tea, often the film with polyphenol has a pH from about 1.5 to about 4.0, and the film(s) with alkaloid and/or amino acid typically has/have a pH from about 4 to about 6, including all ranges subsumed therein.

The specific amount of each biologically active component on the films will vary and be dependent upon the desired dose needed to provide an effective amount of active; however, the generally desired amounts are as defined herein as weight percents based on total weight of the delivery system.

Turning to the FIGURE, the same illustrates a package 10 with a cover 11 for holding or carrying the delivery system for ingestible components 12 of this invention. The delivery system for ingestible components 12 is illustrated to have more than one film, and particularly, three films illustrated as 12a, 12b, and 12c. Such films may be removed individually or in this illustration three at a time by pulling (with fingers not shown) the films 12 through opening 13. It is especially preferred to store and dispense the films (when more than one film is used) in a manner that prevents contamination. Again, and consistent with this invention, more or less than three films may be present, but three are often desired. Moreover, the films may be of any size and shape and the size and shape will typically be determined by the amount of biologically active component that may incorporated within the film and the desired dosage. When more than one water soluble film is employed, such films are not required to be the same size or shape. Usually, the film making up the delivery system for ingestible components is square or rectangular and typically is less than about 65 square centimeters but more than about 2 square centimeters. The thickness of the delivery system for ingestible components varies and often can be as thin as tissue paper and up to about 0.65 centimeters.

When making the films of the delivery system for ingestible components of this invention, formulations with starch, like cornstarch, may be used along with gelatin; proteins, like casein; gums, like carrageenan; and plasticizers, like sorbitol and/or glycerine; emulsifiers; saliva enhancers, like tartaric acid. Typically, the ingredients are added to water and thoroughly mixed. The resulting mixture can than be made into a film on, for example, an endless stainless steel belt system. The film which is dried may than be spooled into rolls for package preparation. Machines like those made commercially available from Servo Systems Co. are illustrative suitable machines for processing the films. When making the films, the biologically active component or components may be added to water along with the ingredients required to make the film. Dependent on the film being used, the pH of the same may be modified with food grade acids and bases. The former typically include, for example, acids like phosphoric, hydrochloric, citric, malic, acetic and benzoic acid and mixtures thereof whereby the latter include, for example, sodium hydroxide, potassium hydroxide, metal carbonates and bicarbonates as well as mixtures thereof.

Other films that may be used include those comprising at least about 90% by weight pulluan. The preferred films, however, are those which comprise 15 to 95% by weight pectin. The most preferred films are those which comprise pectin and at least about 5 to about 15% by weight plasticizer, including ail ranges subsumed therein. Additional information on the films which are suitable for use in this invention may be found in U.S. Application No. 2005/0008735 A1 and U.S. Pat. No. 6,685,978, the disclosures of which are incorporated herein by reference. An often most preferred pectin comprising film suitable for use in this invention is made commercially available by CP Kelco APS and sold under the name GENU®.

In addition to acidulants and bases, other optional additives that may be used in the delivery system of this invention include natural and artificial flavors (especially tea flavors and fruit based flavors), natural and artificial sweeteners, colorants (like the standard food and drug suitable colorants known as FD&C dyes and lakes), vitamins like Vitamin A, D, E, and K as well as Vitamin C, B₁, B₂, B₃, B₆, B₁₂ and folic acid.

Food grade flavors may also be used, if desired, and the same are typically commercially available from suppliers like Wild Flavors, Inc.

There is no limitation with respect to how the delivery system of the present invention is packaged, as long as the package is one which is convenient for the consumer to use. The preferred packaging is depicted, again, in the FIGURE with additional films being stored in storage compartment 14. The films 12 within the storage compartment 14 may simply be stacked on top of each other as individual sheets or they may be “gently” connected to each other by a perforation mechanism. The films 12 need not be the same color and they are not required to have the same taste or fragrance. Typically, when packaged, the films which are identical are placed on top of one another within the packaging to allow for easy dispensing.

The following examples are provided to facilitate an understanding of the present invention. The examples are not intended to limit the scope of the claims.

### EXAMPLE 1

Orally soluble films can be prepared utilizing GENU® Pectin powder made commercially available by CP Kelco U.S., Inc. The desired film was prepared by adding water under conditions consistent wit the supplier's publicly available instructions. The temperature of the ingredients during mixing should be about 35 to about 70° C. The pH values can be adjusted with phosphoric acid. The biologically active component can be mixed in a manner so that the same is homogenously blended in resulting mixtures.

Films may be made by spreading the mixtures on to conventional film making equipment whereby the films that can be made will have a thickness of about 0.5 cm.

Film 1

| Ingredient | Weight Percent |
|---|---|
| GENU ® Pectin | 68 |
| Artificial sweetener | 3 |
| Glycerine | 9.9 |
| Tea polyphenol | 1.3 |
| Caffeine | .3 |
| Theanine | 0.01 |
| Tea powder (excluding actives) | 2.6 |
| Water | to balance |

Film 2

| Ingredient | Weight Percent |
|---|---|
| GENU ® Pectin | 68 |
| Artificial sweetener | 3 |
| Glycerine | 9.9 |
| Tea polyphenol | 4.2 |
| Phosphoric acid | to pH 3.5 |
| Water | to balance |

### Films 3-4

Film 3 can be made in a manner similar to the one described for Film 2 except that Film 3 desirably has 1.8% by weight caffeine in lieu of tea polyphenol.

Film 4 with theanine was made and had 4.2% by weight theanine in lieu of tea polyphenols.

### EXAMPLE 2

Portions of the films prepared in Example 4 were given to panelists. The size of the films given to the panelists delivered biologically active component in an amount consistent with a standard 16 oz. serving of ready-to-drink tea beverage.

All panelists unexpectedly and unanimously concluded that the films making up the delivery system of this invention provided the same positive effects (excluding thirst quenching) associated with consuming a serving of ready-to-drink tea.

What is claimed is:

1. A delivery system for ingestible components comprising:
(a) at least one edible and water soluble film; and
(b) at least one biologically active component
wherein the biologically active component makes up from about 0.015 to about 15 percent by weight of the total weight of the delivery system.

2. The delivery system for ingestible components according to claim 1 wherein the delivery system comprises one edible and water soluble film and at least one biologically active component isolated from tea.

3. The delivery system for ingestible components according to claim 2 wherein the biologically active component comprises alkaloid, amino acid and polyphenol isolated from tea.

4. The delivery system for ingestible components according to claim 1 wherein the delivery system comprises one edible and water soluble film comprising polyphenol and one edible and water soluble film comprising alkaloid and amino acid.

5. The delivery system for ingestible components according to claim 1 wherein the delivery system comprises one edible and water soluble film comprising polyphenol, one edible and water soluble film comprising amino acid and one edible and water soluble film comprising alkaloid.

6. The delivery system for ingestible components according to claim 5 wherein the polyphenol is thearubigin, theaflavin, epigallocatechin gallate, epigallocatechin, epicatechin, epicatehcin gallate and mixtures thereof; the amino acid is theanine and the alkaloids is caffeine, theobromine or a mixture thereof.

7. The delivery system according to claim 4 wherein each water soluble and edible film comprises a color and each water soluble and edible film is a different color.

8. The delivery system according to claim 5 wherein each water soluble and edible film comprises a color and each water soluble and edible film is a different color.

9. The delivery system according to claim 4 wherein each water soluble and edible film has a different pH.

10. The delivery system according to claim 5 wherein each water soluble and edible film has a different pH.

11. A delivery system for ingestible components comprising:
(a) at least two edible and water soluble films; and
(b) at least two biologically active components.

12. The delivery system according to claim 11 wherein the biologically active components are selected isolated from tea.

13. The delivery system according to claim 11 wherein the biologically active components are drugs.

14. The delivery system according to claim 11 wherein each edible and water soluble film comprises a different color or the same color.

15. The delivery system according to claim 11 wherein each edible and water soluble film has the same or different pH.

16. A package comprising the delivery system of claim 11.

17. A method for delivering an ingestible component to a consumer comprising the steps of:
(a) supplying the consumer with the delivery system of claim 11; and
(b) instructing the consumer to consume at least two edible and water soluble films comprising a biologically active component.

18. The method according to claim 17 wherein the consumer is instructed to consume more than one film edible and water soluble film.

19. The method according to claim 17 wherein the biologically active component is isolated from tea.

20. The method according to claim 19 wherein the biologically active component isolated from tea is recovered from tea cream.
